(19) United States
(12) Patent Application Publication
(10) Pub. No.: US 2024/0360063 A1
(43) Pub. Date: Oct. 31, 2024

(54) Selective Aerobic Oxidation of Methane

(71) Applicant: University of Cape Town, Rondebosch, Cape Town (ZA)

(72) Inventors: Eric Wilhelmus Josephus van Steen, Rondebosch (ZA); Junfeng Guo, Rondebosch (ZA); Malefane Gerard Leteba, Rondebosch (ZA); Nasseela Hytoolakhan Lal Mahomed, Rondebosch (ZA); Sinqobile Vuyisile Lusanda Mahlabe, Rondebosch (ZA)

(21) Appl. No.: 18/685,337

(22) PCT Filed: Oct. 26, 2022
(86) PCT No.: PCT/IB2022/060311

§ 371 Date: Feb. 21, 2024

(30) Foreign Application Priority Data
Oct. 27, 2021 (GB) 2115453.9

(51) Int. Cl. C07C 45/33; B01J 21/06; B01J 21/18; B01J 23/42; B01J 23/50; B01J 23/652; B01J 23/89; B01J 35/30; B01J 37/02; B01J 37/08; B01J 37/18; C07C 29/50

(52) U.S. Cl. CPC C07C 45/33; B01J 21/063; B01J 21/18; B01J 23/42; B01J 23/50; B01J 23/6525; B01J 23/892; B01J 35/394; B01J 37/0201; B01J 37/0236; B01J 37/088; B01J 37/18; C07C 29/50

(57) ABSTRACT

A continuous process for aerobic oxidation of methane. The process includes a reactor (10) comprising a bed of a metal-based catalyst (16), a methane feed stream (30) into the reactor (10), an oxygen feed stream (30) into the reactor (10), and a water feed stream (12) into the reactor (10). The methane is oxidised in a single step in the reactor (10) over the metal-based catalyst (16) in the presence of liquid water to produce a reaction product including compounds selected from formaldehyde, oligomers of formaldehyde, methanediol, and products of addition reactions between methanol and formaldehyde, and mixtures thereof.

### INTRODUCTION

This invention relates to a process for selective, aerobic oxidation of methane to formaldehyde and its derived products. In particular, but not exclusively, the invention relates to a continuous process for selective, aerobic oxidation of methane in a single step over a metal-based catalyst in the presence of liquid water, with oxygen as the oxidant.

Formaldehyde and its derived products include formaldehyde, oligomers of formaldehyde (1,3,5 trioxane for example), its hydrated form (methanediol), methanol, and products of addition reactions between methanol and formaldehyde, and mixtures thereof.

### BACKGROUND OF THE INVENTION

Formaldehyde is an important C1-building block for a variety of polymers, which is currently produced from methane involving 3 consecutive, heterogeneously catalysed processes, viz. reforming of methane yielding synthesis gas (Ni-based catalyst at ca. 1000° C.), synthesis gas conversion yielding methanol (Cu/Zn/Al₂O₃ at 250-300° C., p>50 bar) and methanol oxidation yielding formaldehyde (either using a Ag-based catalyst at 600-800° C. or FeMoO₃ at 300-400° C.). A direct route to either methanol or formaldehyde would simplify the overall process and could yield a greater energetic and carbon efficiency.

The search for an efficient process for the direct selective oxidation of methane to formaldehyde and/or methanol is a long-standing challenge, due to the difficulty in selectively activating the methane H₃C—H bond (binding energy of 435 kJ/mol). Methane is a stable, symmetrical, and non-polarisable molecule that is seemingly impervious to nucleophilic and electrophilic attack. In addition, the desired products formed from the selective oxidation of methane, such as formaldehyde and methanol, are much more reactive than methane, rendering them susceptible to consecutive oxidation, yielding the so-called over-oxidation products, CO and CO₂.

The aerobic oxidation of methane can be performed homogeneously at elevated temperatures and pressures, but the radical reaction is difficult to control to obtain consistent high selectivity of the target products, formaldehyde and/or methanol. The early work on heterogeneously-catalysed methane oxidation focused on the use of oxides as the catalyst at ca. 500° C. targeting the formation of C₂-products—ethene/ethane—or formaldehyde.

A number of strategies have been proposed and employed to circumvent the further oxidation of the selective oxidation products, which include the use of protecting agents (i.e., not directly forming the target product, but forming a derivative, for instance converting methane using concentrated sulphuric acid into methane sulfonic acid—this reaction takes place under rather harsh conditions), minimizing the contact of the oxidant with the organic reactant and/or the use of low reaction temperatures in the methane oxidation.

Researchers have developed Fe- and Cu-exchanged zeolites for the selective oxidation of methane to methanol in a circular, non-continuous process, in which the zeolite is activated at high temperature, contacted with methane at an intermediate temperature and subsequently hydrolysed to methanol at low temperature. This process utilises encapsulated multi-nuclear metal complexes on zeolites mimicking biological systems. Attempts to reduce the number of steps to a one-step process is currently hampered by the high reactivity of the product resulting in a strong decrease of the product selectivity upon increasing the conversion.

Batch reactor studies have also shown success in converting methane using H₂O₂ to methyl hydroperoxide (which is subsequently converted to methanol) using either (Cu)Fe-ZSM-5 or AuPd-based nano-alloys. Hydrogen peroxide, however, is an expensive chemical and more expensive than the target product of methanol, although it should be noted that over Pd—Au nano-alloys H₂O₂ can be replaced in part by O₂ or completely by a mixture of H₂+O₂, albeit with a loss in activity.

The activation of methane in the presence of oxygen and carbon monoxide over a supported Rh-catalyst (preferably supported on ZSM-5) has been shown to yield selectively acetic acid. More recently, the selective, aerobic oxidation of methane to methanol has been reported in the presence of light over Fe/TiO₂ (using H₂O₂ as the oxidant) or Au/BP (using O₂ as the oxidant).

The low-temperature, heterogeneous processes operate typically at temperatures where it is difficult to recover the heat of reaction. From an industrial view point a higher reaction temperature would be desired (ca. 200-250° C.).

Methane activation has been studied theoretically to a significant extent. Is has been proposed that the selective activation of methane and, hence, the formation of methanol may be achieved by the direct interaction of methane with surface atomic oxygen that is pre-adsorbed on a metal surface avoiding the formation of metal-alkyl surface species, which have been identified as precursors for the over-oxidation products. The mechanism proposed implies that surfaces should be saturated with oxygen-containing species so that methane will only interact with the adsorbed layer rather than with the metal surface.

Group 10 and 11 metals, or suitable alloys thereof, have been identified as potential catalysts for the selective oxidation of methane, provided that their surface can be saturated with surface, oxygen-containing species to prevent the interaction of methane with the bare surface. This is not achieved readily. For instance, nickel, palladium and copper may oxidise under the targeted reaction conditions, and silver is known to form an oxide overlayer only reaching a saturation coverage of 0.5 ML, depending on the oxygen chemical potential. Similarly, the saturation coverage of oxygen on Pt(111) is known to be limited to ca. 0.44 ML.

It is an object of this invention to alleviate at least some of the above-mentioned problems associated with existing processes for the selective oxidation of methane to formaldehyde and its derived products.

It is a further object of this invention to provide a process that will be a useful alternative to existing processes.

### SUMMARY OF THE INVENTION

In accordance with a first aspect of the invention there is provided a continuous process for aerobic oxidation of methane, the process including:
a) providing a reactor comprising a bed of a metal-based catalyst;
b) providing a methane feed stream into the reactor;
c) providing an oxygen feed stream into the reactor; and
d) providing a water feed stream into the reactor;
wherein the methane is oxidised in a single step in the reactor over the metal-based catalyst in the presence of liquid water to produce a reaction product including compounds selected from formaldehyde, oligomers of formaldehyde (1,3,5 trioxane for example), methanediol, and products of addition reactions between methanol and formaldehyde, and mixtures thereof.

Preferably, the metal of the metal-based catalyst is selected from the group consisting of Pt, Pd, Ni, Ag and Au, and alloys thereof, wherein the alloyed metal is selected from the group consisting of Pt, Pd, Ni, Cu, Ag and Au.

More preferably, the metal-based catalyst is a platinum-based or platinum-alloy-based catalyst.

In one embodiment, the metal-based catalyst includes a support which is selected from the group consisting of titania, alumina, silica and other refractory oxides, and carbon.

In another embodiment, the catalyst is selected from the group consisting of Pt/TiO₂(P25), Pt/TiO₂(rutile), Pt(Mo)/TiO₂(rutile), Pt/Al₂O₃ and Pt/C.

In yet another embodiment, the catalyst is selected from the group consisting of Pt/Au, Pt/Ag and Pt/Cu alloys, supported on alumina or titania.

Preferably, the support is titania.

In a further embodiment, the reactor is a trickle-bed reactor.

In yet a further embodiment, the reactor operating temperature is between about 150° C. and about 250° C.

In yet another further embodiment, the reactor total operating pressure is between about 20 bar and about 100 bar.

In a preferred embodiment, heat is recovered from the reactor.

In accordance with a second aspect of the invention there is provided a continuous process for aerobic oxidation of methane, the process including:
a) providing a reactor comprising a bed of a metal-based catalyst;
b) providing a methane feed stream into the reactor;
c) providing an oxygen feed stream into the reactor; and
d) providing a water feed stream into the reactor;
wherein the methane is oxidised in a single step in the reactor over the metal-based catalyst in the presence of liquid water to produce a reaction product including compounds selected from formaldehyde, oligomers of formaldehyde, methanediol, and products of addition reactions between methanol and formaldehyde, and mixtures thereof, with a selectivity of the compounds or mixtures thereof of over 70%.

### BRIEF DESCRIPTION OF THE FIGURES

Non-limiting embodiments of the invention will now be described by way of example only and with reference to the following figures:

FIG. 1: TEM images of platinum-based catalysts for methane oxidation comprising:
Left: Incipient wetness impregnated 10 wt.-% Pt/TiO₂(P25) reduced at 400° C.,
Middle: Incipient wetness impregnated 10 wt.-% Pt/TiO₂(rutile) reduced at 400° C., and
Right: Colloid impregnated 10 wt.-% Pt(Mo)/TiO₂(rutile);

FIG. 2: Platinum-based nanoparticles (NPs) used as precursors for metal-based catalysts for methane oxidation;

FIG. 3: A schematic drawing of a trickle-bed reactor for methane oxidation in accordance with this invention;

FIG. 4: Methane oxidation as a function of temperature over Pt/TiO₂-WI-R (inlet partial pressures—see Table 2):
Top: Methane conversion as a function of temperature,
Middle: Selectivity for selective oxidation products (non-CO and non-CO₂), and
Bottom: 1,3,5-Trioxane content in the fraction of selective oxidation;

FIG. 5: Methane oxidation as a function of temperature over Pt/TiO₂-NP (inlet partial pressures —CH₄: 1.2-1.6 bar; O₂: 1.3-1.8 bar; m_{catalyst}=0.15 g):
Top: Methane conversion as a function of temperature, and
Bottom: Selectivity for selective oxidation products (non-CO and non-CO₂);

FIG. 6: Methane oxidation as a function of temperature over Pt/TiO₂-NP (inlet partial pressures—see Table 3; m_{catalyst}=0.5 g):
Top: Methane conversion as a function of temperature, and
Bottom: Selectivity for selective oxidation products (non-CO and non-CO₂);

FIG. 7: Methane oxidation as a function of temperature over Ag/TiO₂-NP (inlet partial pressures—see Table 4; m_{catalyst}=0.39 g, Ag loading=9.4 wt. %):
Top: Methane conversion as a function of temperature, and
Bottom: Selectivity for selective oxidation products (non-CO and non-CO₂);

FIG. 8: Methane oxidation as a function of temperature over Pt₃Ag/TiO₂-NP (inlet partial pressures—see Table 5; m_{catalyst}=0.152 g, 10 wt % metal loading);

FIG. 9: Methane conversion over colloid impregnated 10 wt.-% Pt_NPs/TiO₂ in a gas-phase methane oxidation reactor (W/F_{CH4,0}=4.1 g_{cat}·min/mmol);

FIG. 10: Methane conversion and product selectivity over 10 wt. % (a) Pt/rutile and (b) Pt/P-25 catalysts in presence of liquid water at 220° C. and 30 bar (PCH₄=0.5 bar, PO₂=1.5 bar, Psat,H₂O=23.1 bar);

FIG. 11: Catalyst activity of various impregnated platinum catalysts on different supports in the selective oxidation of methane;

FIG. 12: Selectivity for the formation of formaldehyde (or methanol in the case of Pt/TiO₂ (P25)) over various impregnated platinum catalysts on different supports in the selective oxidation of methane

FIG. 13: Performance of Pt₃Ni/TiO₂ (rutile) in a trickle bed reactor.

### DETAILED DESCRIPTION OF THE INVENTION

The present invention will now be described more fully hereinafter with reference to the accompanying figures, in which some, but not all embodiments of the invention are shown.

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. The terminology includes the words specifically mentioned above, derivatives thereof, and words of similar import. It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

As used herein, the term selective oxidation products means products obtained from oxidation of methane, different from CO or CO₂, and the term oxygenates has the same meaning.

The following examples are offered by way of illustration and not by way of limitation.

### Catalyst Synthesis

The metal-based catalysts illustrated herein may be synthesised either by incipient wetness or by colloidal impregnation of Pt-nanoparticles on a titania support (either rutile or P25). It will be appreciated that Pd, Ni, Cu, Ag and Au, or alloys thereof, may similarly be used, as alternatives for Pt, and that Pt or platinum alloys are illustrated here as non-limiting examples of a metal base for the catalyst. The person of ordinary skill in the art would have a reasonable expectation that the invention could be carried out using other metals or alloys thereof, such that, but not limited to, catalytically active materials will remain in the metallic (zero-valent) state during the reaction, or that metals may be selected from group 9 to 11 metals, or alloys thereof. Without thereby wishing to be bound by any particular theory, it is believed that the catalytic activity can be tuned by manipulating the d-band centre of the surface metal atoms by an appropriate selection of alloying elements. It will also be appreciated that carbon or other refractory oxides may be used as suitable alternatives for titania, including, but not limited to, alumina and silica, and that titania is illustrated here as one non-limiting example of a support for the catalyst. Without thereby wishing to be bound by any particular theory, a refractory oxide may be selected on the basis of the surface acidity or basicity of the support, and it is believed that less basic and acidic sites will lead to improved catalytic performance.

### Example 1

Incipient wetness impregnation involved contacting a support of TiO₂ (either rutile or P25) with a solution of platinic acid (H₂PtCl₆) to obtain 10 wt.-% platinum on the support. The solid was dried, calcined at 400° C. (air flow rate: 48 mlₙ/min/g) and subsequently reduced at either 400° C. (H₂ flow rate: 48 mlₙ/min/g) for 5 hrs or 600° C. in flowing hydrogen. Platinum is then in the metallic state as confirmed using XRD. The resulting average particle size for Pt/TiO₂(P25) was 4.5 nm contacted and ca. 9 nm for Pt/TiO₂(rutile). The active metal dispersion was measured using static H₂-chemisorption performed at 80° C. in the pressure range 0.1-600 mm Hg and determined to be 8.9% for Pt/TiO₂(P25) and 9.1% for Pt/TiO₂(rutile).

### Example 2

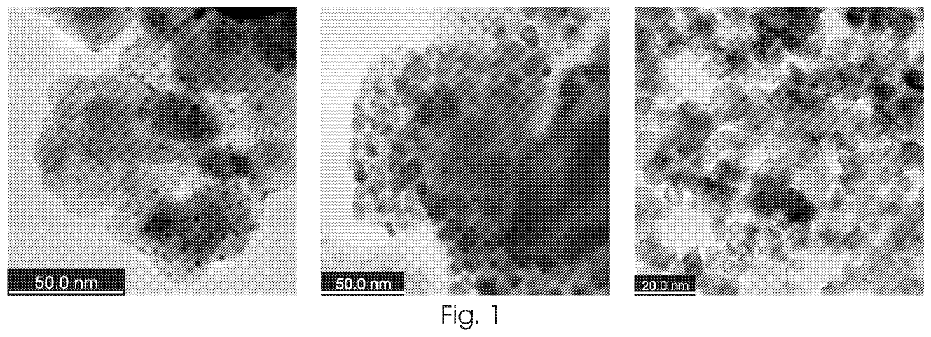

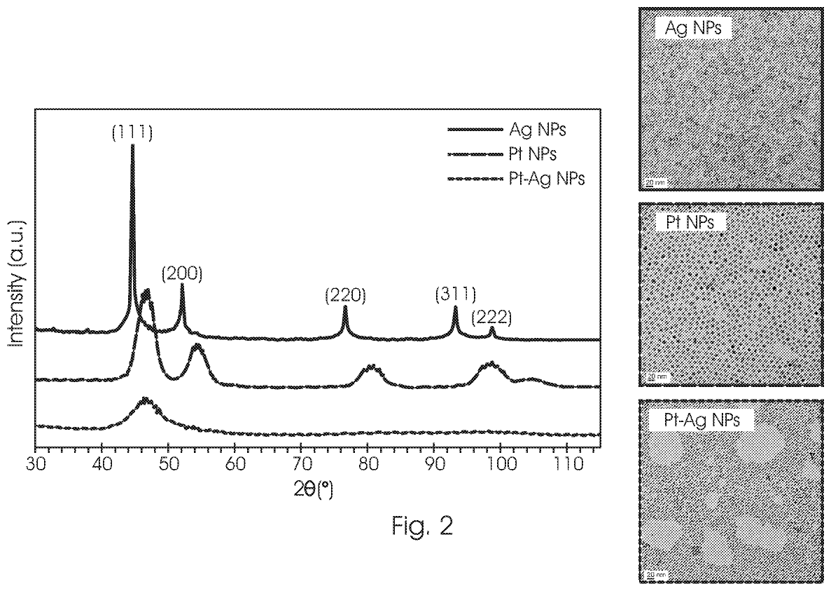

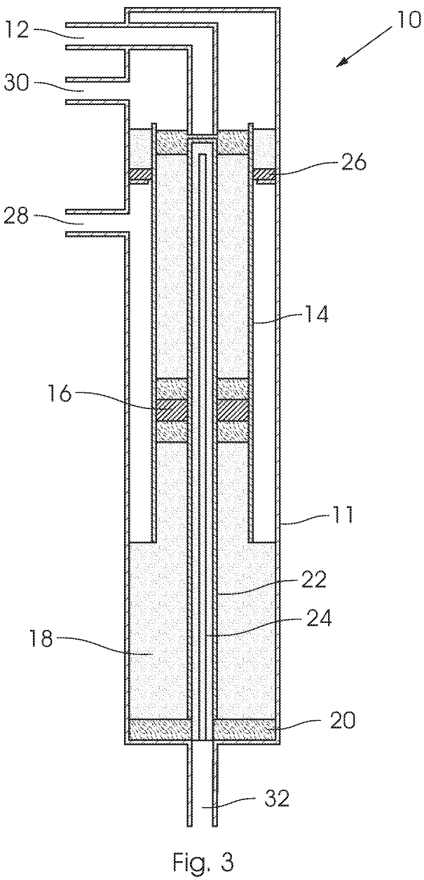

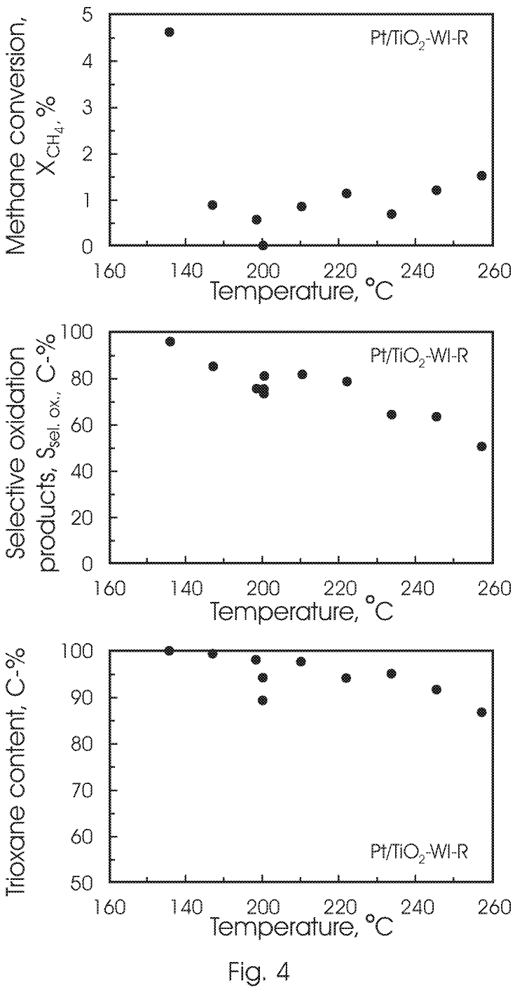

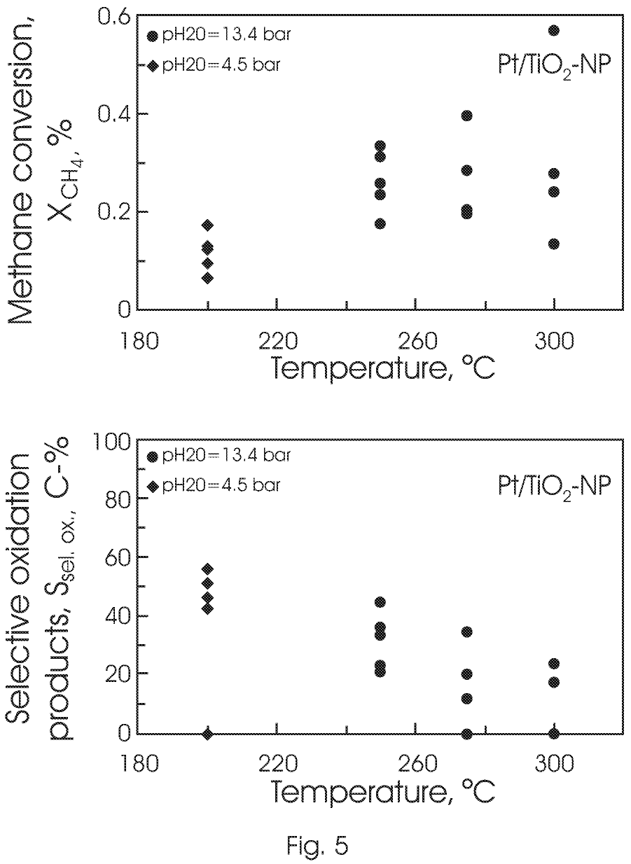

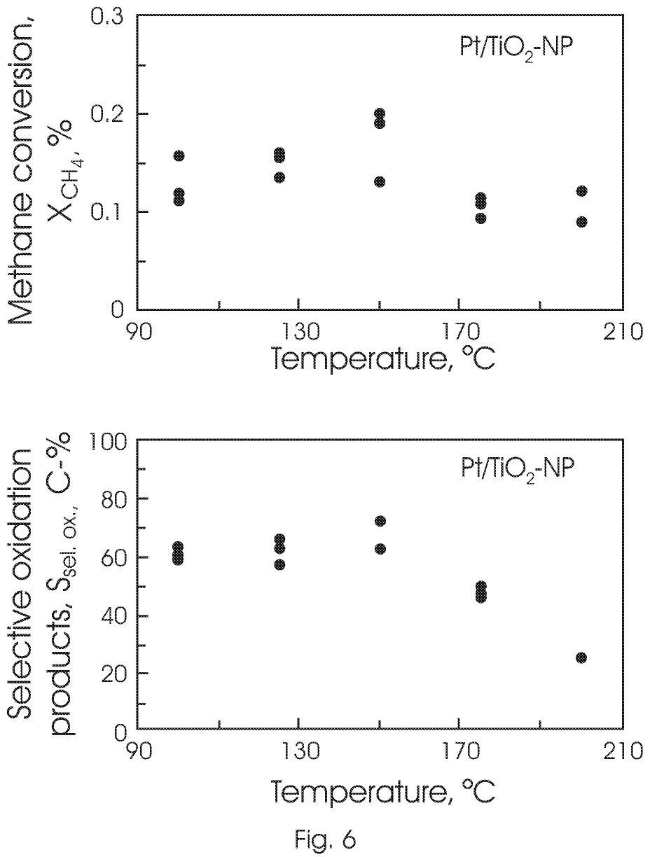

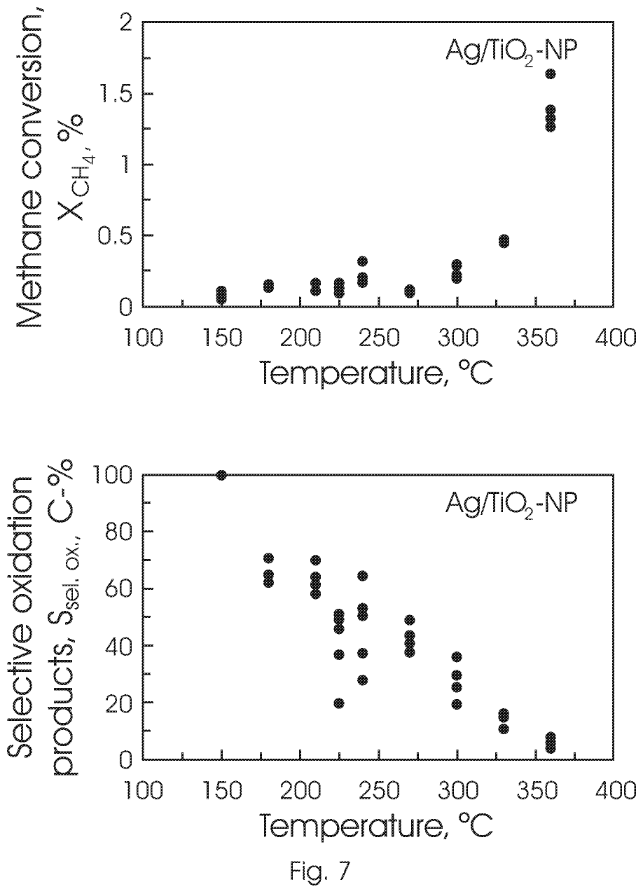

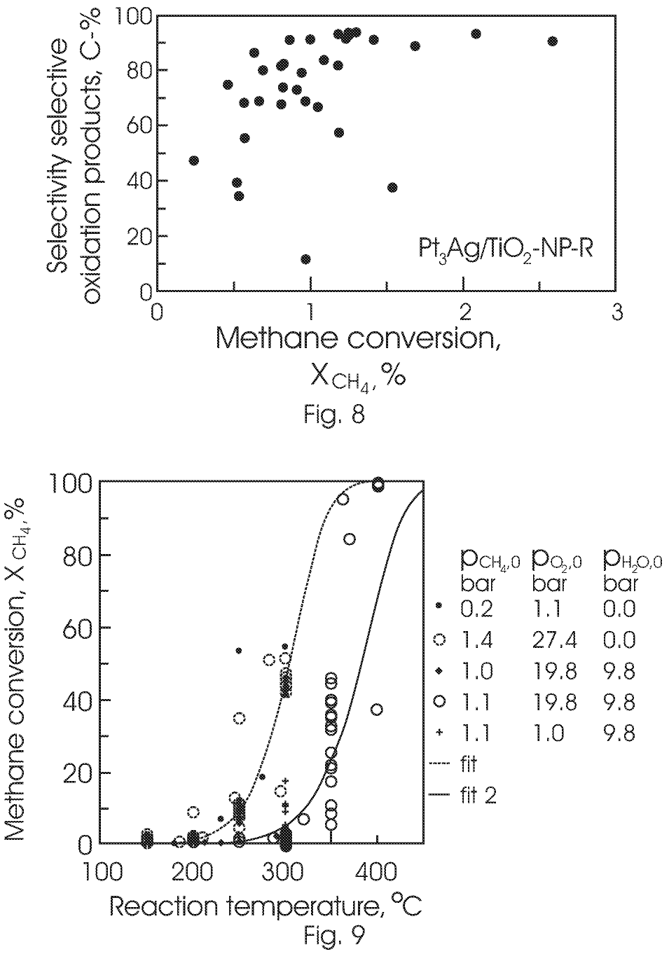

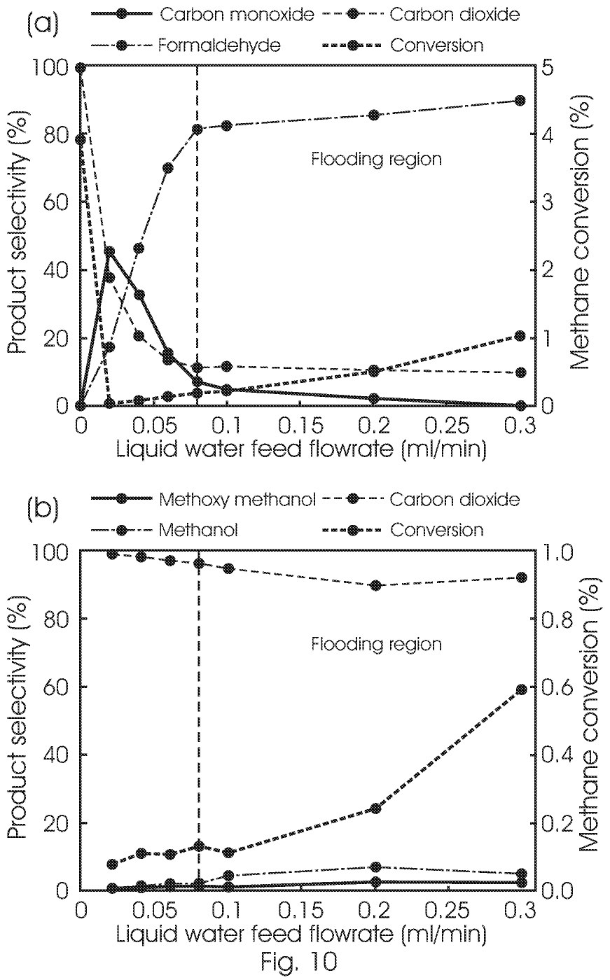

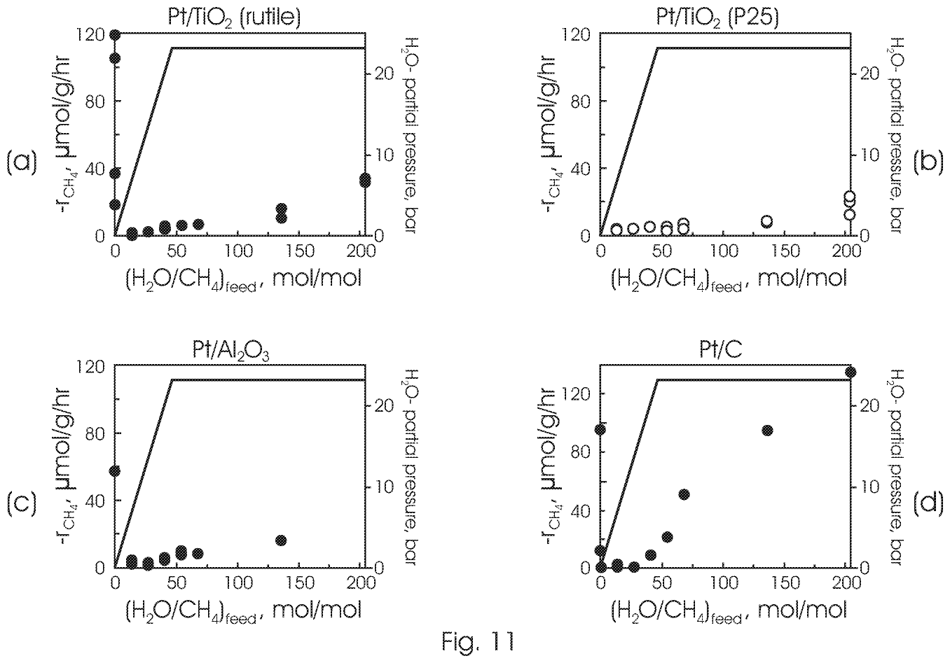

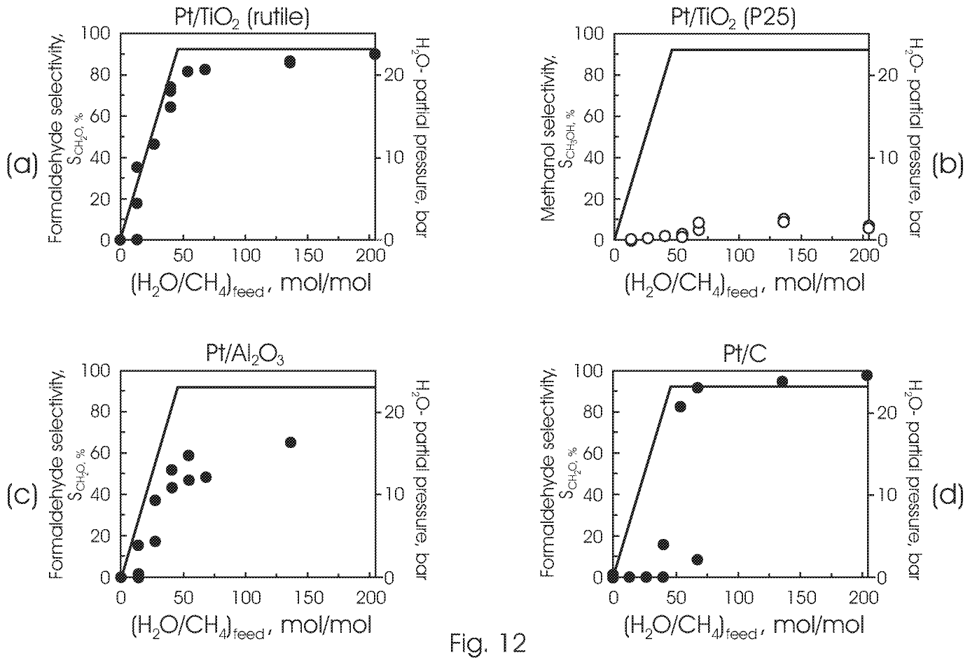

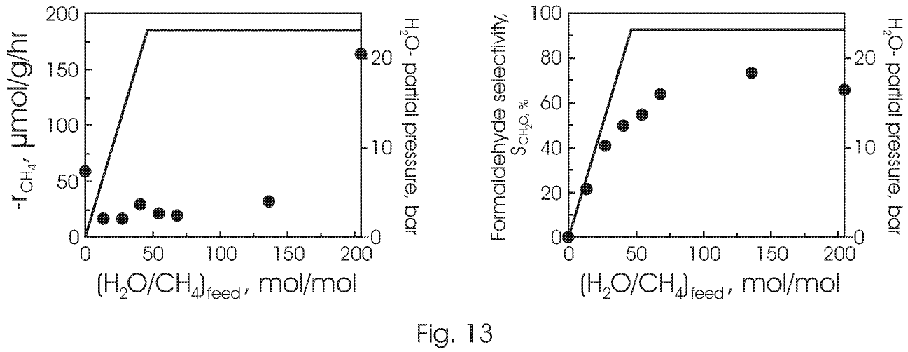

Monometallic platinum nanoparticles (NPs) were synthesized in a one-neck round-bottom flask: platinum acetylacetonate (0.12 g, 0.31 mmol) was dissolved via vigorous stirring at 150° C. in a ternary mixture of surfactants oleylamine (20 ml), trioctylamine (20 ml) and hexadecylamine (5 g), in the presence of high boiling point solvent benzyl ether (25 ml). The resultant pale-yellow homogeneous solution was heated up to 200° C. with a heating rate of 10° C./min. The solution rapidly turned dark brown and was maintained at this temperature for 5 min, removed from the heat source and cooled down to room temperature. The black product was finally re-dispersed in chloroform, yielding a dark brown colloidal suspension. The separation-purification-resuspension processes of the as-prepared colloidal solution were repeated several times to remove all the unwanted solvents and surface-unbound surfactants. TEM images (shown in FIG. 2) show monodisperse nanoparticles with a narrow particle size distribution and mean particle diameter of less than 5 nm. The Pt nanoparticles display a near-spherical shape.

### Example 3

A second batch of monometallic platinum nanoparticles was synthesised using Mo(CO)₆ as the reduction agent. The precursor salt Pt(acac)₂ (0.16 g, 0.41 mmol) was dissolved in oleyl amine (20 ml), oleic acid (20 ml) and benzyl ether (25 ml). The resulting metal salt-surfactant-solvent reaction mixture was heated at 150° C. for 5 to 10 minutes under vigorous magnetic stirring in a round bottom flask. Upon addition of Mo(CO)₆ (0.1 g, 0.38 mmol), the resultant pale-yellow homogeneous solution turned dark purple with evolving cloudy smoke. The bulk organic synthesis mixture then turned dark brown during the heat-up process to 200° C., with a heating rate of 10° C./min. The resultant colloidal mixture was held at this temperature for 10 to 15 min. Thereafter, the colloidal medium was removed from the heat source and quenched using cold water to ensure no structural transformations during the cooling process. Subsequently, the as-synthesized nanoparticles were extracted from the synthesis media through flocculation by adding excess absolute ethanol. After settling (typically 1 to 2 days), the excess organic solvents were decanted and the particles were further cleaned by re-suspending in absolute ethanol. This colloidal refining process was performed 3 times. The black product was finally re-suspended in chloroform, yielding a dark brown colloidal suspension for Pt NPs. TEM images show monodisperse nanoparticles with a narrow particle size distribution and mean particle diameter of less than 5 nm. The Pt nanoparticles display a near-spherical shape. Elemental analysis showed no molybdenum in the catalyst.

### Example 4

A binary platinum-silver nano-alloy with a nominal composition of Pt₃Ag was synthesized by dissolving Pt(acac)₂ (0.16 g, 0.41 mmol) and AgNO₃ (0.023 g, 0.14 mmol) in a mixture of oleyl amine (20 ml), oleic acid (20 ml) and benzyl ether (25 ml). The resulting metal salt-surfactant-solvent reaction mixture was heated at 150° C. for 5 to 10 minutes under vigorous magnetic stirring in a round bottom flask. Upon addition of Mo(CO)₆ (0.1 g, 0.38 mmol), the resultant pale-yellow homogeneous solution turned dark purple with evolving cloudy smoke. The bulk organic synthesis mixture then turned dark brown during the heat-up process to 200° C., with a heating rate of 10° C./min. The resultant colloidal mixture was held at this temperature for 10 to 15 min. Thereafter, the colloidal medium was removed from the heat source and quenched using cold water to ensure no structural transformations during the cooling process. Subsequently, the as-synthesized NPs were extracted from the synthesis media through flocculation by adding excess absolute ethanol. After settling (typically 1 to 2 days), the excess organic solvents were decanted and the particles were further cleaned by re-suspending in absolute ethanol. This colloidal refining process was performed 3 times. The resulting nano-alloy has a worm-like structure (see FIG. 2). The XRD-pattern of the Pt—Ag alloy is dominated by the (111) reflection of the FCC-structure showing facile growth along the dense (111) plane. The composition according to Vegard's law is Pt:Ag=0.95:0.05. The nanoparticles were supported on TiO₂(rutile) by colloidal impregnation.

### Example 5

For comparison, silver nanoparticles were synthesized by dissolving AgNO₃ (0.13 g, 0.77 mmol) in oleyl amine (20 ml), oleic acids (20 ml) and benzyl ether (25 ml). The resulting metal salt-surfactant-solvent reaction mixture was heated at 150° C. for 5 to 10 minutes under vigorous magnetic stirring in a round bottom flask. Upon addition of Mo(CO)₆ (0.1 g, 0.38 mmol), the resultant pale-yellow homogeneous solution turned dark purple with evolving cloudy smoke. The bulk organic synthesis mixture then turned dark brown during the heat-up process to 200° C., with a heating rate of 10° C./min. The resultant colloidal mixture was held at this temperature for 10 to 15 min. Thereafter, the colloidal medium was removed from the heat source and quenched using cold water to ensure no structural transformations during the cooling process. Subsequently, the as-synthesized NPs were extracted from the synthesis media through flocculation by adding excess absolute ethanol. After settling (typically 1 to 2 days), the excess organic solvents were decanted and the particles were further cleaned by re-suspending in absolute ethanol. This colloidal refining process was performed 3 times. The black product was finally re-suspended in chloroform, yielding a dark reddish colloidal suspension for Ag NPs.

Herein, catalysts are referred to according to the active metal, support, and method of synthesis or morphology. For instance, a platinum catalyst synthesised via the solution-phase synthetic approach is referred to as Pt/TiO₂-NP (NP—nanoparticles), Pt/TiO₂-NW (NW—nanowires) and Pt/TiO₂-WI-R (WI—wet impregnation, R—rutile). As used herein, the term Pt(Mo) means a platinum-based catalyst synthesised using Mo(CO)₆ as a reductant.

### Reactor Setup and Analysis

The reactor (10) may be a fixed-bed reactor, more particularly a trickle-bed reactor, comprising a bed of metal-based catalysts (16) as described hereinbefore. Feed streams may be provided continuously into the reactor, comprising a methane feed stream, an oxygen feed stream and a water feed stream. The reactor may comprise further feed streams including Helium and Argon. Importantly, the oxidation of methane over the metal-based catalyst (16) may take place continuously in the presence of water in the liquid phase. The operation of the reactor (10) may be described by way of the following example.

By way of a non-limiting example, trickle-bed conditions in which the invention may be carried out, at 250° C., include water partial pressure of ca. 40 bar, CH₄ partial pressure of ca. 30 bar, and a stoichiometric amount of O₂ at a partial pressure of 15 bar, totalling a minimum of 85 bar.

### Example 6

The trickle-bed reactor (10) consisted of a quartz tube (14) 38 cm long and 12 mm in inside diameter packed with ca. 1.5 g of pelletized and sieved (to 150-250 μm particles) Pt/TiO₂ catalyst (16) in its centre. The void space on top of the catalyst bed (16) was packed with silicon carbide particles (18) (diameter ˜300 μm). The catalyst (16) and the silicon carbide (18) were held in place with 2 small glass (quartz) wool plugs (20) on either end. A quartz sheathed (22) thermocouple (24) was placed at the centre of the catalyst bed (16) for temperature measurement. The reactor body (11) is enclosed in an aluminium heating furnace controlled with multiple heating zones controlled by thermocouples on the outside of the reactor (10). The isothermal zone in the reactor (10) was ca. 10 cm. The quartz tube (14) was placed inside a 19.05 mm outside diameter stainless steel tube. An O-ring (26) (made from fluorocarbon for example) may be placed between the stainless-steel tube and quartz tube (14), located between an inert gas (Argon for example) inlet port (28) and a feed gas inlet port (30) comprising methane, oxygen and helium.

Liquid water was fed into the trickle bed reactor (10) via a liquid water inlet port (12) and mixed with oxygen, helium and methane at the top of the reactor (taking care to avoid the formation of water droplets at the entrance point of the water). The flow rates of methane, oxygen helium and liquid water were precisely controlled.

Argon flowed, pressure-controlled, through the annular space between the two tubes at the same pressure as inside the quartz tubing (14). The bottom of the reactor (10) rested on a bed of silicon carbide (18) (diameter ˜300 μm; bed length 15.5 cm) to ensure evaporation of the liquid water dripping out of the catalyst bed (16) (the argon flow rate was typically set at ca. 2-3 times the total flow rate through the catalyst bed (16)). The temperature of the bottom zone was adjusted depending on the water flow rate to achieve smooth operation. The final mixture was expanded to 1 bar over a needle valve. The argon was allowed to mix with the reaction product exiting the quartz tubing (14) at the end of the reactor (10), via a reactor outlet port (32), to dilute it prior to its expansion to 1 bar. This, and due to the small size of the reactor, was done to minimize risks associated with the flammability of methane and oxygen mixtures as well as allowing the reactor (10) to be operated at elevated pressures (<100 bar).

Reaction products were measured and analysed online using a GC-Methanizer-FID system. Methane is the only carbon containing compound fed into the reactor. The conversion, yield and selectivity can be estimated directly from the GC-trace and assuming a quantitative conversion in the methanizer and that there is no loss in carbon:

$${Y}_{i} ( c - \% ) = \frac{Peak area of i}{Total peak area} \times 100 ( C - \% )$$

The methane conversion was calculated as the sum of the yields:

$${X}_{{CH}_{4}} = \sum {Y}_{i} ( C - \% ) = ( 1 - \frac{Peak area of {CH}_{4}}{Total peak area} ) \times 100 ( C - \% )$$

The selectivity for the formation of compound i was calculated from the yield of compound i and the methane conversion:

$${S}_{i} ( c - \% ) = \frac{{Y}_{i}}{{X}_{{CH}_{4}}} \times 100 ( C - \% )$$

Table 1 shows the products from the selective oxidation of methane as identified via GC-FID and their corresponding retention times.

TABLE 1
Retention times corresponding to the product obtained
during the selective oxidation of methane.

| Retention time (min) | Component | Chemical Formula |
|---|---|---|
| 1.6 | Carbon monoxide | CO |
| 1.7 | Methane | CH₄ |
| 2.1 | Carbon dioxide | CO₂ |
| 3.2 | Ethane | C₂H₆ |
| 5.5 | Formaldehyde | CH₂O |
| 6.2 | Methanediol | |
| 6.9 | Methanol | CH₃OH |
| 7.8 | Methyl formate | C₂H₄O₂ |
| 9.2 | Dimethylether | C₂H₆O |
| 9.5 | Methoxymethanol | CH₃OCH₂OH |
| 10.05 | Dimethoxymethane | CH₃OCH₂OCH₃ |
| 10.9 | Formic acid | CH₂O₂ |
| 11.4 | Dimethylcarbonate | CH₃OCOCH₃ |
| 13.36 | Methylhydroperoxide | CH₃OOH |
| 14.1 | Trioxane | CH₂OCH₂OCH₂O |
| 14.98 | Paraformaldehyde | HO(CH₂O)ₙH* |

*n = 8-100

A gas-phase reactor was set up for comparative purposes, and had a similar setup and design philosophy as the above trickle bed reactor, with the differences described as follows. Helium was fed into the reactor through an evaporator, acting as an inert diluent and carrier of water, which was continuously pumped into the evaporator at a constant flow rate using an HPLC-pump. The temperature in the evaporator was regulated to be below the boiling point of water at the pressure of the reactor at the top of the evaporator and ca. 20° C. above the boiling point of water at this pressure at the end of the evaporator to ensure smooth evaporation. Quantities of helium, methane and oxygen were mass flow-controlled. The water-helium mixture was mixed with the oxygen-methane mixture at the top of the reactor. The reactor consisted of a quartz tube 25 cm long and 2.4 mm in inside diameter packed with ca. 400 mg of the pelletized and sieved Pt/TiO₂ catalyst in its centre. The void space on top of the catalyst was packed with silicon carbide particles (diameter ˜300 μm). The catalyst and the silicon carbide were held in place with 2 small glass (quartz) wool plugs on either end. A quartz sheathed thermocouple was placed at the bottom of the catalyst bed for temperature control. The quartz tube was placed inside a 6.35 mm outside diameter stainless steel tube.

### Continuous Selective Aerobic Oxidation of Methane

The continuous, selective, aerobic oxidation of methane as described herein may be exercised over a number of process parameters, using the aforementioned metal-based catalysts, as illustrated by the examples that follow.

### Example 7

The invention may be exercised using platinum impregnated on the rutile form of titania (m_{catalyst}=0.36 g; 9.2 wt.-% Pt) as catalyst over the process parameters shown in Table 2.

TABLE 2
Feed gas flow rates and partial pressures employed during the
oxidation of methane to oxygenates over platinum impregnated
on the rutile form of titania (m_{catalyst} = 0.36 g; 9.2 wt.-% Pt)

| Feed | Flowrate | Partial |
|---|---|---|
| component | (ml(NTP) · min⁻¹) | Pressure^{a} (Bar) |
| He | 77.0 | 9.8 |
| O₂ | 16.0 | 1.8 |
| CH₄ | 2.5 | 0.3 |
| H₂O | 0.05 (fed as a liquid) | 8.1 |

^{a}Partial pressure at the inlet of the reaction zone (saturation pressure of water at lowest reaction temperature, 176° C., 9.15 bar)

FIG. 4 shows the performance of methane oxidation of the impregnated platinum on the rutile form of titania at a relatively low partial pressure of methane and oxygen upon co-feeding water. The initially obtained conversion at a low temperature is high but drops rapidly upon increasing the reaction temperature. This anomalous behaviour is related to the change in the composition of the coverage of the catalyst surface and a consequence of the start-up procedure. Low conversion of ca. 0.01% was obtained at a reaction temperature of 200° C., which was obtained after the methane oxidation at 250° C. (the original conversion at 200° C. was 0.57%).

The obtained conversion of ca. 0.5% corresponds to platinum-time-yield of 0.2 hr⁻¹ (mol CH₄ converted per hr per mole of Pt), which would correspond to a turnover frequency of 0.8 hr¹ if the catalyst has a dispersion of 25%.

The catalyst is selective for the formation of selective oxidation products, with a selectivity of more than 70 C-% at temperatures below 220° C. even when the catalyst had deactivated. The major selective oxidation product obtained with this catalyst was trioxane, although with increasing reaction temperature the fraction of the product identified as trioxane decreases (and a product appearing in the GC trace at a retention time of 9.2 min—which has been identified as dimethylether—was observed). Trioxane is an important chemical, serving as an intermediate in the production of acetal resins (polyoxymethylenes), which find application in the production of plastics.

### Example 8

On-purpose synthesized platinum nanoparticles supported on the rutile form of titania with a defined particle size of platinum of ca. 4 nm were utilised for the methane oxidation. A partial pressure of methane of ca. 1.2-1.6 bar and a partial pressure of O₂ between 1.3 and 1.8 bar was utilised at a reaction temperature between 20° and 300° C. At these conditions, the reaction was performed in the gas phase, instead of at trickle bed conditions, for comparative purposes. From FIG. 5, although still effective, the catalyst activity is somewhat lower than the activity obtained over platinum impregnated on the rutile form of titania. The selectivity towards selective oxidation products is reduced (with both trioxane and the dimethylether being formed; the selectivity towards trioxane is less than the one obtained over platinum impregnated on the rutile form of titania). This shows the importance of liquid water at trickle bed reaction conditions, in other words flooding at the catalyst.

### Example 9

The invention was carried out over platinum nanoparticles supported on the rutile form of titania at low reaction temperatures, as shown in Table 3.

TABLE 3
Feed gas flow rates and partial pressures at the various reaction
temperatures employed during the oxidation of methane to
oxygenates over platinum nanoparticles supported on the rutile
form of titania at low reaction temperatures.

| | | Partial Pressure (Bar) | | | | |
|---|---|---|---|---|---|---|
| Feed | Flowrate | 100° | 125° | 150° | 175° | 200° |
| components | (ml · min⁻¹) | C. | C. | C. | C. | C. |
| He | 34 | 14.9 | 13.8 | 11.9 | 9.9 | 9.9 |
| O₂ | 5 | 2.2 | 2.0 | 1.7 | 1.5 | 1.5 |
| CH₄ | 4.5 | 2.0 | 1.8 | 1.6 | 1.3 | 1.3 |
| H₂O | 0.02 (as a liquid) | 1.0* | 2.3* | 4.8* | 7.3 | 7.3 |

*liquid water in the reactor as well (flooding)

As can be seen from FIG. 6, although still effective, the conversion of methane is not very high. The selectivity for selective oxidation products (in this case mainly dimethylether), increases with decreasing reaction temperature, plateaus at ca. 60 C-% under conditions where flooding takes place.

### Example 10

Silver nanoparticles supported on the rutile form of titania (Ag/TiO₂-NP-R) were utilised in the oxidation of methane over a wide range of temperatures at the conditions given in Table 4.

TABLE 4
Feed gas flow rates and partial pressures employed during the
oxidation of methane to oxygenates over Ag/TiO₂-NP-R.

| | Flowrate | Partial |
|---|---|---|
| Feed gas | (ml(NTP) · min⁻¹) | pressure (bar) |
| He | 149.6 | 8.3 |
| O₂ | 16.8 | 0.9 |
| CH₄ | 2.0 | 0.1 |
| H₂O | 0.01 (fed as liquid) | 0.7 |

As can be seen from FIG. 7, the conversion increases with increasing temperature. The conversion-temperature behaviour shows two distinctly different regions in an Arrhenius diagram, viz. a low temperature region with an activation energy of ca. 19 kJ/mol and a high temperature region (T>240° C.) with an activation energy of ca. 46 kJ/mol. The selectivity for selective oxidation products (mainly dimethylether) decreases with increasing temperature (and increasing methane conversion), indicating that reaction temperatures should not be excessively high such that complete oxidation is dominant.

### Example 11

The invention was carried out with platinum-silver alloy nanoparticles supported on the rutile form of titania (Pt₃Ag/TiO₂-NP-R) as catalyst for the oxidation of methane at 225° C. at the conditions given in Table 5.

TABLE 5
Feed gas flow rates and partial pressures employed
during the oxidation of methane to oxygenates
over platinum-silver alloy nanoparticles supported
on the rutile form of titania (Pt₃Ag /TiO₂-NP-R)

| | Flowrate | Partial |
|---|---|---|
| Feed gas | (ml · min⁻¹) | Pressure (Bar) |
| He | 32 | 3.2 |
| O₂ | 12 | 1.2 |
| CH₄ | 2.5 | 0.3 |
| H₂O | 0.12 | 15.4 |

As can be seen from FIG. 8, the conversion varied between 0.2 and 2.6%. Alloying platinum with silver resulted in an improvement in the selectivity towards formaldehyde, which was the only selective oxidation product obtained.

### Example 12

Platinum nanoparticles supported on the rutile form of titania (Pt(Mo)/TiO₂) were utilised in the oxidation of methane at the conditions indicated in Table 6, which also indicates the conversion and selectivity results.

TABLE 6
Effect of trickle bed conditions in the gas-phase methane
oxidation reactor on the selective methane oxidation over
10%-Pt(Mo)/TiO₂ (reaction performed at 20 bar
with a constant flow rate of CH₄ at 4.5 mlₙ/min, O₂ at
5.0 mlₙ/min, He at 34.0 mlₙ/min, H₂O(I) at 0.02 ml/min)

| Reaction | | | | |
|---|---|---|---|---|
| temperature, ° C. | 100 | 125 | 150 | 175* |
| p_{CH4, inlet}, bar | 2.0 | 1.8 | 1.6 | 1.3 |
| p_{O2, inlet}, bar | 2.2 | 2.0 | 1.7 | 1.5 |
| p_{He, inlet}, bar | 14.9 | 13.8 | 11.9 | 9.9 |
| p_{H2O,sat}, bar | 1.0 | 2.3 | 4.8 | 7.3 |
| X_{CH4}, % | 0.13 ± 0.02 | 0.15 ± 0.01 | 0.17 ± 0.04 | 0.10 ± 0.01 |
| -r_{CH4}, μmol/g/hr | 143 ± 22 | 166 ± 11 | 188 ± 44 | 110 ± 11 |
| S_{CO2}, C-% | 40.1 ± 1.9 | 38.9 ± 4.3 | 34.9 ± 5.5 | 53.3 ± 1.6 |
| S_{dimethylether}, C-% | 56.5 ± 3.9 | 61.1 ± 4.3 | 51.0 ± 18.7 | 46.7 ± 1.6 |
| Sₜᵣᵢₒₓₐₙₑ, C-% | 3.3 ± 5.8 | — | 14.0 ± 24.2 | — |

*Under these conditions a single fluid phase is expected.

### Example 13

The rate of reaction and conversion level of methane is independent of the O₂ and CH₄ partial pressure or feed rate, as can be seen from FIG. 9 in which the invention was carried out over 10 wt.-% Pt/TiO₂(rutile)-NP at the reaction parameters indicated in the figure, in a gas-phase reactor. Upon flooding the reactor, at reaction temperatures below the saturation point of water, the valuable products of formaldehyde, oligomers of formaldehyde (1,3,5 trioxane for example), methanediol, methanol, and the add-products between methanol and formaldehyde were obtained.

### Example 14

CH₄ conversion and product selectivity over 10 wt. % (a) Pt/rutile and (b) Pt/P-25 catalysts in the presence of liquid water at 220° C. and 30 bar (PCH₄=0.5 bar, PO₂=1.5 bar, Psat,H₂O=23.1 bar) can be seen in FIG. 10.

For the Pt/rutile catalyst, CH₄ conversion decreases drastically from 3.9% without water to 0.04% at a water flowrate of 0.02 ml/min, which is attributed to the metal surface being covered by hydroxyl species impeding CH₄ activation. For both catalysts, the initial increase in the water partial pressure steadily improves CH₄ conversion, while strongly favouring the formation of oxygenates and thus suppressing CO/CO₂ selectivity. Compared to Pt/P-25, Pt/rutile showed better catalytic activity and highest oxygenate selectivity with 90% of formaldehyde. In the flooding regime (feed water flowrate >0.08 ml/min) as shown in FIG. 10(a), the presence of liquid water does not have considerable effect on the formaldehyde selectivity as increases slowly until it reaches a plateau.

Importantly, in examples 7 to 14, the presence of liquid water at the catalytically active site (i.e. flooding) favours activity as well as selectivity towards selective oxidation products. See for example FIG. 10(a), and the conversion, reaction rate and selectivity changes in Table 6 between trickle bed conditions at 150° C. and the single, vapour phase conditions at 175° C.

Operating the reactor at higher temperatures, for example between 150° C. and 250° C., allows for the recovery of heat from the reactor. This aids in making the process more economically viable.

### Example 15

The invention was carried out with platinum impregnated on TiO₂ (rutile), TiO₂ (P25), Al₂O₃ and C. The catalysts were prepared by incipient wetness impregnation of platinic acid in deionized water on titanium dioxide (Rutile: S_{BET}=44.5 m²/g; P25: S_{BET}=46.4 m²/g), alumina (γ-Al₂O₃; S_{BET}=73.9 m²/g) or carbon (S_{BET}=232 m²/g) as a support. The support was contacted with an aqueous solution of platinic acid (H₂PtCl₆) to obtain 10 wt.-% platinum on the support. The solid was dried, calcined at 400° C. (air flow rate: 48 mlₙ/min/g) and subsequently reduced for 5 hrs at 400° C. in flowing hydrogen (48 mlₙ/min/g). The activity over the various impregnated platinum catalysts on different supports in the selective oxidation of methane can be seen in FIG. 11, at reaction conditions of F_{CH4,0}/W˜ 3.2 mmol/min/hr; p_{CH4, inlet}=0.5 bar; p_{O2, inlet}=1.5 bar; pₜₒₜₐₗ=30 bar. The activity achieved with Pt/C is notably high, also achieving a conversion of ca. 4% at the high water to methane ratio.

FIG. 12 shows the selectivity for the formation of formaldehyde as a function of the inlet ratio of water to methane, in a trickle bed reactor at reaction conditions of F_{CH4,0}/W˜3.2 mmol/min/hr; p_{CH4, inlet}=0.5 bar; p_{O2, inlet}=1.5 bar; pₜₒₜₐₗ=30 bar; T=220° C. High selectivity for the formation of formaldehyde (90-98%) can be obtained using supports such as TiO₂ in its rutile form, or carbon. The reaction over Pt/TiO₂(P25) yielded some methanol, and no formaldehyde.

### Example 16

The invention was carried out using Pt₃Ni on a TiO₂ (rutile) support in a trickle bed reactor at reaction conditions of F_{CH4,0}/W˜4.9 mmol/min/hr; p_{CH4, inlet}=0.5 bar; p_{O2, inlet}=1.5 bar; pₜₒₜₐₗ=30 bar; T=220° C. The results can be seen in FIG. 13, indicating good activity of 163 μmol/g/hr and a conversion of 3.3% at the highest water feed rate. The selectivity had a maximum of ca. 73%.

It will be appreciated that the above examples are only some embodiments of the invention and that there may be many variations without departing from the spirit and/or the scope of the invention. It is easily understood from the present application that the particular features of the present invention, as generally described and illustrated in the figures, can be arranged and designed according to a wide variety of different configurations. In this way, the description of the present invention and the related figures are not provided to limit the scope of the invention but simply represent selected embodiments.

The skilled person will understand that the technical characteristics of a given embodiment can in fact be combined with characteristics of another embodiment, unless otherwise expressed or it is evident that these characteristics are incompatible. Also, the technical characteristics described in a given embodiment can be isolated from the other characteristics of this embodiment unless otherwise expressed.

1. A continuous process for aerobic oxidation of methane, the process including:
a) providing a reactor comprising a bed of a metal-based catalyst;
b) providing a methane feed stream into the reactor;
c) providing an oxygen feed stream into the reactor; and
d) providing a water feed stream into the reactor;
wherein the methane is oxidised in a single step in the reactor over the metal-based catalyst in the presence of liquid water to produce a reaction product including compounds selected from formaldehyde, oligomers of formaldehyde, methanediol, and products of addition reactions between methanol and formaldehyde, and mixtures thereof.

2. The process according to claim 1, wherein the metal of the metal-based catalyst is selected from the group consisting of Pt, Pd, Ni, Ag and Au, and alloys thereof, wherein the alloyed metal is selected from the group consisting of Pt, Pd, Ni, Cu, Ag and Au.

3. The process according to claim 1, wherein the metal of the metal-based catalyst is selected from the group consisting of Pt and Ag, and alloys thereof, wherein the alloyed metal is selected from the group consisting of Pt, Pd, Ni, Cu, Ag and Au.

4. The process according to claim 1, wherein the metal-based catalyst is a platinum-based or platinum-alloy-based catalyst.

5. The process according to claim 1, wherein the metal-based catalyst includes a support which is selected from the group consisting of titania, alumina, silica and other refractory oxides, and carbon.

6. The process according to claim 1, wherein the catalyst is selected from the group consisting of Pt/TiO₂(P25), Pt/TiO₂(rutile), Pt(Mo)/TiO₂(rutile), Pt/Al₂O₃ and Pt/C.

7. The process according to claim 1, wherein the catalyst is selected from the group consisting of Pt/Au, Pt/Ag and Pt/Cu alloys, supported on alumina or titania.

8. The process according to claim 7, wherein the support is titania.

9. The process according to claim 1, wherein the reactor is a trickle-bed reactor.

10. The process according to claim 1, wherein the reactor operating temperature is between about 150° C. and about 250° C., and the reactor total operating pressure is between about 20 bar and about 100 bar.

11. The process according to claim 1, wherein heat is recovered from the reactor.

12. A continuous process for aerobic oxidation of methane, the process including:
a) providing a reactor comprising a bed of a metal-based catalyst;
b) providing a methane feed stream into the reactor;
c) providing an oxygen feed stream into the reactor; and
d) providing a water feed stream into the reactor;
wherein the methane is oxidised in a single step in the reactor over the metal-based catalyst in the presence of liquid water to produce a reaction product including compounds selected from formaldehyde, oligomers of formaldehyde, methanediol, and products of addition reactions between methanol and formaldehyde, and mixtures thereof, with a selectivity of the compounds or mixtures thereof of over 70%.
